(19) United States
(12) Patent Application Publication
(10) Pub. No.: US 2021/0065414 A1
(43) Pub. Date: Mar. 4, 2021

(54) SYSTEM AND METHOD FOR SINOGRAM SPARSIFIED METAL ARTIFACT REDUCTION

(71) Applicant: THE GENERAL HOSPITAL CORPORATION, Boston, MA (US)

(72) Inventor: Synho Do, Lexington, MA (US)

(21) Appl. No.: 16/644,777

(22) PCT Filed: Sep. 7, 2018
(86) PCT No.: PCT/US18/49929

§ 371 Date: Mar. 5, 2020

Related U.S. Application Data
(60) Provisional application No. 62/555,717, filed on Sep. 8, 2017.

(51) Int. Cl. G06T 11/00; G06T 7/11; G06T 7/00; A61B 6/03; A61B 6/00

(52) U.S. Cl. CPC G06T 11/008; G06T 11/006; G06T 7/11; G06T 7/0014; G06T 2207/20224; A61B 6/5258; G06T 2207/10081; G06T 2207/30004; A61B 6/032

(57) ABSTRACT

Described her are systems and methods for reconstructing images from x-ray attenuation data (e.g., sinogram data) in which metal artifacts are reduced. The algorithms described in the present disclosure take advantage of accurate forward system modeling and one or more iterative reconstruction techniques (IRTs) (e.g., those using compressed sensing) to reconstruct images from incomplete data sets. Rather than replace measurements that are identified as corrupted with inaccurate ones, the systems and methods described in the present disclosure exclude those corrupted measurements in the fidelity term of the energy functional. As a result, the corrupted measurements are not included in the image formation process. In doing so, the reconstruction problem is changed from being about inaccurate data correction to sparse data image reconstruction.

### CROSS-REFERENCE TO RELATED APPLICATIONS

This application claims the benefit of U.S. Provisional Patent Application Ser. No. 62/555,717, filed on Sep. 8, 2017, and entitled “Sinogram Sparsified Metal Artifact Reduction Technique (SSMART),” which is herein incorporated by reference in its entirety.

### BACKGROUND

In recent years, there have been significant improvements made in two different techniques to combat the problem of metal artifacts in CT imaging: improving the algorithm in itself and adding spectral information to improve the quality of the image. Metal Artifact Reduction (MAR) is a technique that can be used when dealing with patients who have metallic implants in their bodies. The MAR technique is a constant necessity for scanning baggage in the security field, where the presence and amount of metal is much more frequent and severe.

One of the drawbacks of MAR is the nonlinear effects of measurements caused by the corruption or shifting of the energy spectra. It has been speculated that MAR is a problem without a simple, generalized solution, and the solutions currently used are limited to the correction of mild artifacts and local artifacts.

There is a need, therefore, to provide techniques for eliminating or otherwise reducing metal artifacts in CT imaging.

### SUMMARY OF THE DISCLOSURE

The present disclosure addresses the aforementioned drawbacks by providing a method for reconstructing an image of a subject using a computed tomography (CT) system, which in some instances may include an electron beam computed tomography (EBCT) system or a multi-detector CT (MDCT) system. The method includes providing to a computer system, data acquired from a subject using a CT system; reconstructing a first image from the provided data using the computer system; generating a metal component mask from the first image using the computer system, wherein the metal component mask depicts regions in the subject containing metal; and generating masked data with the computer system by using the metal component mask to remove ray sums in the provided data that pass through the regions in the subject containing metal. A second image is reconstructed from the masked data using the computer system; a difference image is generated with the computer system by computing a difference between the first image and the second image; and corrected data are generated with the computer system by forward projecting the difference image to generate segmented artifact data and by computing a difference between the provided data and the segmented artifact data. A third image is then reconstructed from the corrected data using the computer system.

It is another aspect of the present disclosure to provide a computer system for reconstructing an image from data acquired with a CT system, which in some instances may include an EBCT system or an MDCT system. The computer system incudes one or more processors and a memory having stored thereon instructions that when executed by the one or more processors cause the one or more processors to perform the steps comprising: (a) accessing data acquired from a subject using a CT system; (b) reconstructing a first image from the provided data; (c) generating a metal component mask from the first image, wherein the metal component mask depicts regions in the subject containing metal; (d) generating masked data by using the metal component mask to remove ray sums in the accessed data that pass through the regions in the subject containing metal; (e) reconstructing a second image from the masked data; (f) generating a difference image by computing a difference between the first image and the second image; (g) generating segmented artifact data by forward projecting the difference image; (h) generating corrected data by computing a difference between the accessed data and the segmented artifact data; and (i) reconstructing a third image from the corrected data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 shows the steps of an example algorithm for reconstructing images according to some embodiments described in the present disclosure.

FIG. 2 is a flowchart setting forth the steps of an example method for reconstructing an image from data acquired using a CT system, which in some embodiments may be an EBCT system or an MDCT system.

FIG. 3 is an example system model of an EBCT system.

FIG. 4 is a block diagram of an example EBCT system.

FIGS. 5A and 5B illustrate an example CT system, which in some instances may be configured as an MDCT system.

### DETAILED DESCRIPTION

Described here are systems and methods for reconstructing images from x-ray attenuation data (e.g., sinogram data) in which metal artifacts are reduced. The algorithms described in the present disclosure take advantage of accurate forward system modeling and one or more iterative reconstruction techniques (IRTs) (e.g., those using compressed sensing) to reconstruct images from incomplete data sets. Rather than replace measurements that are identified as corrupted with inaccurate ones, the systems and methods described in the present disclosure exclude those corrupted measurements in the fidelity term of the energy functional. As a result, the corrupted measurements are not included in the image formation process. In doing so, the MAR problem is changed from being about inaccurate data correction to sparse data image reconstruction.

The systems and methods described in the present disclosure thus provide for the reconstruction of images from sparse sinogram data acquired with an x-ray imaging system, such as a CT system, in which metal artifacts, including streaking artifacts, and low frequency shadowing artifacts are significantly reduced, even high clutters cases. In some instances, the CT system may be an EBCT system. In some other instances, the CT system may be an MDCT system.

As stated, the systems and methods described in the present disclosure remove less reliable ray-sums in the fidelity term of an iterative image reconstruction. As an example, the less reliable ray-sums can be those ray-sums that pass through metal components. These metal passed ray-sums go through beam hardening, spectral shifting, intensity clipping effects, and so on. It is challenging to correct for all of these effects with the measurements from an energy integration detector. The systems and methods described in the present disclosure, however, provide a technical advantage of being able to reconstruct higher quality images from data containing metal passed ray-sums. As such, using these techniques can improve the use of EBCT systems or other CT systems that implement energy integration detectors.

The systems and methods described in the present disclosure implement a decision rule process to determine metal passed ray-sums on the image domain and an image reconstruction method to reconstruct an image without metal component and additional artifacts. In such a reconstruction, the number of measurements can be smaller than the number of unknowns, making it an under-determined problem. Therefore, a sinogram sparsified reconstruction technique is implemented.

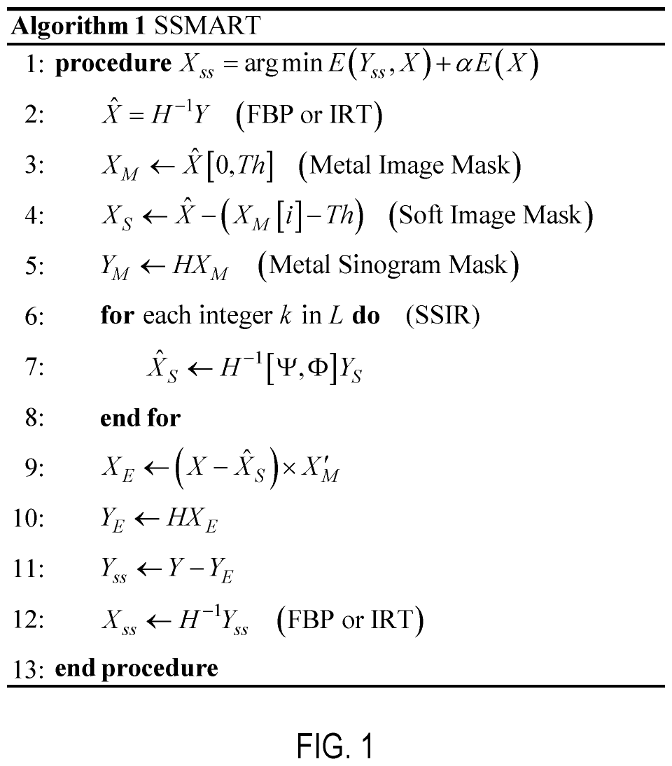

As will be described below in more detail, the systems and methods described in the present disclosure can implement an image reconstruction technique that includes pre-correction steps and post-compensation steps with a sinogram sparsified iterative reconstruction (SSIR). One non-limiting example of a reconstruction technique that can be implemented is generally illustrated in FIG. 1, in which steps 2-5 correspond to pre-correction steps and steps 9-11 correspond to post compensation steps.

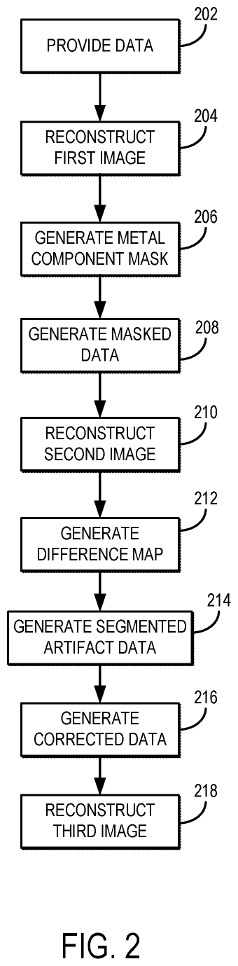

Referring now to FIG. 2, a flowchart is illustrated as setting forth the steps of an example method for reconstructing an image from data acquired using a computed tomography (CT) system, which may in some embodiments be an EBCT system or an MDCT system. The method includes providing data to a computer system, as indicated at step 202. In general, the data are x-ray attenuation data. In some embodiments, the data includes sinogram data. Providing the data can include retrieving previously acquired data from a memory or other suitable data storage, or can also include acquiring data with a CT system and providing the acquired data to the computer system.

A first image is reconstructed from the data, as indicated at step 204. In general, the first image can be reconstructed using any suitable image reconstruction technique. As one example, the first image can be reconstructed using a reconstruction technique that implements a least-squares (LS) solution calculated by using whole ray-sums on the sinogram data. In some embodiments, the LS solution can be a filtered back projection (FBP) type image reconstruction algorithm, which may include an Xrec reconstruction technique or any other suitable analytical reconstruction method.

A metal component mask is then generated from the first image, as indicated at step 206. Generating a metal component mask can include determining a threshold value based on the signal intensities associated with metal components that are depicted in the first image. The threshold value can be manually selected based on a visual inspection of the first image, can be selected based on a predetermined value, can be determined by processing the first image, or so on. As one non-limiting example, the threshold value can be 0.1. In general, the metal component mask will be a binary image. For instance, pixels associated with a metal component can be assigned a value of one (or zero) and other pixels assigned a value of zero (or one).

Masked data is created from the metal component mask, as indicated at step 208. Preferably, the masked data represents data associated with the metal components present in the subject being imaged. Creating the masked data can include forward projecting the metal component mask onto the sinogram domain. As one example, the masked data can be generated by making a binary decision regarding the presence of metal in a certain location of the first image as represented by the metal component mask. As another example, the masked data can be generated by applying the metal component mask to the first image (e.g., by pixel-wise multiplying the metal component mask and the first image) and then forward projecting the resulting masked image.

A second image is reconstructed from the masked data, as indicated at step 210. The reconstruction can include a preprocessing step in which ray-sums that pass through a metal component are removed using the masked data. As one example, reconstructing the second image can include implementing an iterative reconstruction. The iterative reconstruction may be based on a Haar wavelet transform. As another example, the iterative reconstruction can be a sinogram-sparsified image reconstruction that implements an iterative shrinking algorithm. For example, the following iterative reconstruction implementing an iterative shrinking algorithm can be used,

\hat{x}=arg min ½∥y−Ax∥²+πρ(x) (1);

where x is an image, y is a sinogram, A is a system matrix, λ is a weighting parameter, and ρ(x) can be given as,

$$ρ ( x ) = \vert x \vert - s log ( 1 + \frac{\vert x \vert}{s} ) ; \qquad ( 2 )$$

which leads to a near L1-norm for small values of s>0. As one example, s can be s=0.0001. The system matrix, A, can be selected as A=H[Ψ,Φ], where Ψ and Φ are two n×n unitary matrices and H is a forward system matrix. Using this, the iterative reconstruction algorithm represented by Eqn. (1) can be rewritten as,

\hat{x}=arg min ½∥y−H(Ψx_{Ψ}−Φx_{Φ})∥²+λρ(x_{Ψ})+λρ(x_{Φ}) (3).

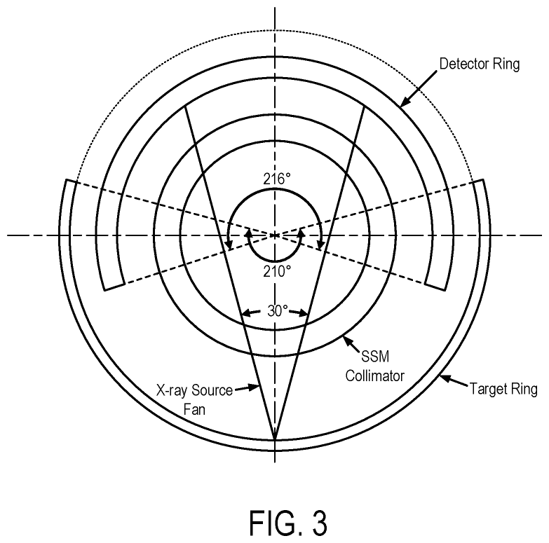

CT systems, including EBCT systems, MDCT system, or otherwise, can have multiple sinogram formats in the pre-processing stages; thus, the system model used in the system matrix, A, should be appropriately selected based on the sinogram format for the CT system. As one example, when the CT system used is an EBCT system the system model can be based on a native geometry model of EBCT, an example of which is shown in FIG. 3. Of the two concentric half circles in FIG. 3, the larger circle depicts an electron beam target (source ring), while the smaller circle represents the detector modules. The radius of the source ring in this example is 900.0 mm, and the detector ring, which contains 864 channel detector modules that measure over 216 degree, has a 676.0 mm radius. The reconstruction field of view is a 475.0 mm circle. This example system can collect full sinogram data within 116.16 ms (total sweep time) without any gantry motion.

A difference map is created using the first and second images, as indicated at step 212. As one example, the difference map can be generated by subtracting the first image and the second image. In some embodiments, generating the difference map includes subtracting the first and second images and then multiplying the result with the metal component mask. The difference map isolates metal artifacts within the image.

Segmented artifact data is generated from the difference map, as indicated at step 214. The segmented artifact data can be generated, for example, by forward projecting the difference map onto the sinogram domain. The segmented artifact data generally represents the metal artifacts found in the second image. Corrected data are then generated using the original data and the segmented artifact data, as indicated at step 216. As an example, the corrected data can be generated by subtracting the segmented artifact data from the original data (e.g., using an element-wise subtraction).

A third image is reconstructed from the corrected data, as indicated at step 218. The reconstruction method can be any suitable image reconstruction technique, including a FBP reconstruction or other suitable analytical reconstruction, or an iterative reconstruction, such as a sinogram-sparsified image reconstruction, iterative shrinkage algorithm, or so on.

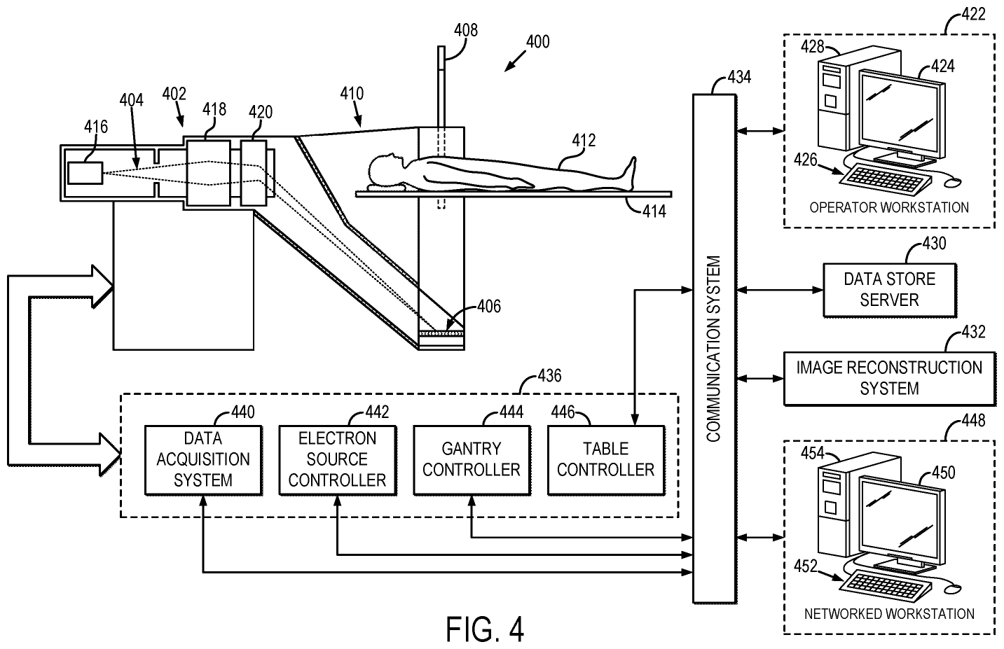

Referring now to FIG. 4, an example of an electron beam computed tomography (“EBCT”) imaging system 400, which can be implemented in some embodiments described in the present disclosure, is illustrated. The EBCT imaging system 400 includes an electron source assembly 402 that generates an electron beam 404 that is projected onto one or more target rings 406. When the electron beam 404 impinges upon the one or more target rings 406, x-rays are generated and directed toward a detector array 408. The one or more target rings 406 and the detector array 408 are coupled to a rotatable gantry 410, such that the one or more target rings 406 and the detector array 408 can be rotated about a subject 412, such as a medical patient or an object undergoing examination, that is positioned on a table 414.

The electron source assembly 402 includes an electron source 416, which may be an electron gun, one or more focusing coils 418, and one or more bending coils 420. The electron source 416 generates an electron beam 404 that extends through the one or more focusing coils 418 where the electron beam 404 is focused onto the one or more target rings 406. The one or more bending coils 420 bend or otherwise deflect the electron beam 404 so that it impinges upon the one or more target rings 406. Additionally, the one or more bending coils 420 can be operated to rapidly sweep the electron beam 404 along the surface of the one or more target rings 406. For example, the one or more target rings 406 can be serially, or otherwise, scanned in order to provide for multiple different imaging sections.

The one or more target rings 406 are generally partially circular, or otherwise curved. When the electron beam 404 impinges on the one or more target rings 406 an x-ray beam is generated and directed towards the detector array 408. The x-ray beam may be, for example, a planar beam. A portion of the x-ray beam, which may be a fan-shaped portion, is detected by the detector array 408 after passing through the subject 412. The data measured by the detector array 408 are utilized to reconstruct a tomographic image of the subject 412, as described in the present disclosure.

In general, the detector array 408 can be in the form of a ring. In some instances, the detector array 408 is semicircular and may extend over an angular range, such as 210 degrees. The target rings 406 and detector array 408 can be at least partially overlapped. For instance, the target rings 406 and detector array 408 can be overlapped such that at least 180 degrees of projection data can be obtained.

Together, the x-ray detector elements in the detector array 408 sense the projected x-rays that pass through the subject 412. Each x-ray detector element produces an electrical signal that may represent the intensity of an impinging x-ray beam and, thus, the attenuation of the x-ray beam as it passes through the subject 412. In some configurations, each x-ray detector element is capable of counting the number of x-ray photons that impinge upon the detector. During a scan to acquire x-ray projection data, the gantry 410 and the components mounted thereon rotate about an isocenter of the EBCT system 400.

The EBCT system 400 also includes an operator workstation 422, which typically includes a display 424; one or more input devices 426, such as a keyboard and mouse; and a computer processor 428. The computer processor 428 may include a commercially available programmable machine running a commercially available operating system. The operator workstation 422 provides the operator interface that enables scanning control parameters to be entered into the EBCT system 400. In general, the operator workstation 422 is in communication with a data store server 430 and an image reconstruction system 432. By way of example, the operator workstation 422, data store sever 430, and image reconstruction system 432 may be connected via a communication system 434, which may include any suitable network connection, whether wired, wireless, or a combination of both. As an example, the communication system 434 may include both proprietary or dedicated networks, as well as open networks, such as the internet.

The operator workstation 422 is also in communication with a control system 436 that controls operation of the EBCT system 400. The control system 436 generally includes a data acquisition system (“DAS”) 440, an electron source controller 442, a gantry controller 444, and a table controller 446. The electron source controller 442 provides power and timing signals to the electron source assembly 402, and the table controller 446 is operable to move the table 414 to different positions and orientations within the EBCT system 400. The electron source controller 442 can receive instructions from the operator workstation 422 that control the focusing and bending of the electron beam 404.

The rotation of the gantry 410 is controlled by the gantry controller 444, which controls the rotation of the gantry 410 about an axis of rotation. In response to motion commands from the operator workstation 422, the gantry controller 444 provides power to motors in the EBCT system 400 that produce the rotation of the gantry 410. For example, a program executed by the operator workstation 422 generates motion commands to the gantry controller 444 to move the gantry 410, and thereby the target rings 406 and detector array 408, in a prescribed scan path.

The DAS 440 samples data from the one or more x-ray detectors in the detector array 408 and converts the data to digital signals for subsequent processing. For instance, digitized x-ray data is communicated from the DAS 440 to the data store server 430. The image reconstruction system 432 then retrieves the x-ray data from the data store server 430 and reconstructs an image therefrom. The image reconstruction system 432 may include a commercially available computer processor, or may be a highly parallel computer architecture, such as a system that includes multiple-core processors and massively parallel, high-density computing devices. Optionally, image reconstruction can also be performed on the processor 428 in the operator workstation 422. Reconstructed images can then be communicated back to the data store server 430 for storage or to the operator workstation 422 to be displayed to the operator or clinician.

The EBCT system 400 may also include one or more networked workstations 448. By way of example, a networked workstation 448 may include a display 450; one or more input devices 452, such as a keyboard and mouse; and a processor 454. The networked workstation 448 may be located within the same facility as the operator workstation 422, or in a different facility, such as a different healthcare institution or clinic.

The networked workstation 448, whether within the same facility or in a different facility as the operator workstation 422, may gain remote access to the data store server 430, the image reconstruction system 432, or both via the communication system 434. Accordingly, multiple networked workstations 448 may have access to the data store server 430, the image reconstruction system 432, or both. In this manner, x-ray data, reconstructed images, or other data may be exchanged between the data store server 430, the image reconstruction system 432, and the networked workstations 448, such that the data or images may be remotely processed by the networked workstation 448. This data may be exchanged in any suitable format, such as in accordance with the transmission control protocol (“TCP”), the Internet protocol (“IP”), or other known or suitable protocols.

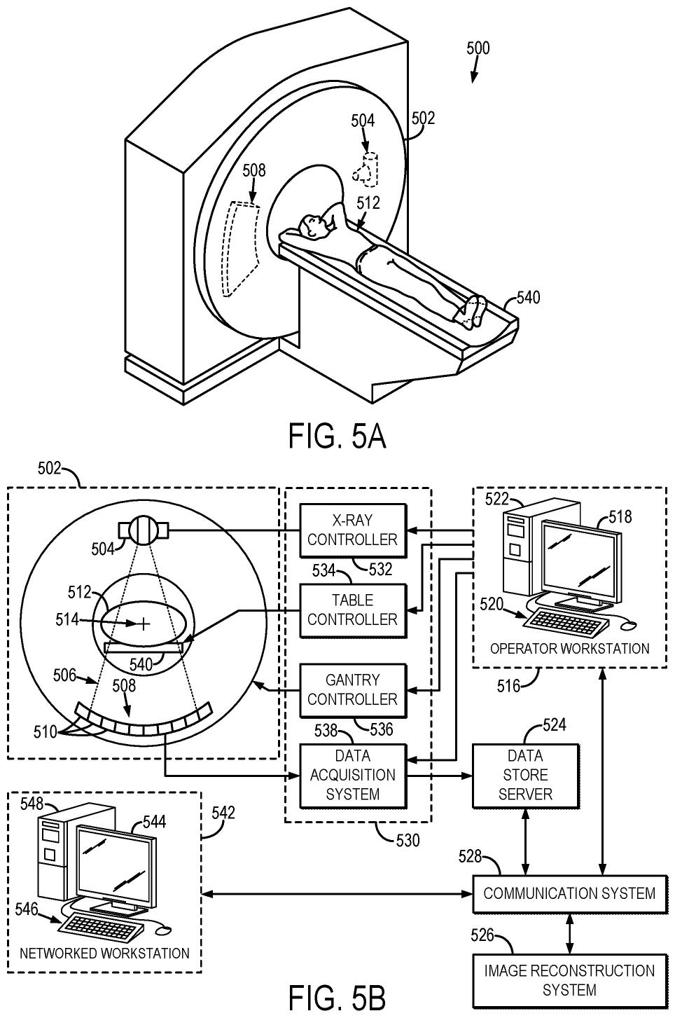

Referring particularly now to FIGS. 5A and 5B, an example of an x-ray computed tomography (“CT”) imaging system 500 is illustrated. The CT system includes a gantry 502, to which at least one x-ray source 504 is coupled. The x-ray source 504 projects an x-ray beam 506, which may be a fan-beam or cone-beam of x-rays, towards a detector array 508 on the opposite side of the gantry 502. The detector array 508 includes a number of x-ray detector elements 510. In some configurations the detector array 508 can be a multi-detector array, such that the CT system 500 is an MDCT system. Together, the x-ray detector elements 510 sense the projected x-rays 506 that pass through a subject 512, such as a medical patient or an object undergoing examination, that is positioned in the CT system 500. Each x-ray detector element 510 produces an electrical signal that may represent the intensity of an impinging x-ray beam and, hence, the attenuation of the beam as it passes through the subject 512. In some configurations, each x-ray detector 510 is capable of counting the number of x-ray photons that impinge upon the detector 510. During a scan to acquire x-ray projection data, the gantry 502 and the components mounted thereon rotate about a center of rotation 514 located within the CT system 500.

The CT system 500 also includes an operator workstation 516, which typically includes a display 518; one or more input devices 520, such as a keyboard and mouse; and a computer processor 522. The computer processor 522 may include a commercially available programmable machine running a commercially available operating system. The operator workstation 516 provides the operator interface that enables scanning control parameters to be entered into the CT system 500. In general, the operator workstation 516 is in communication with a data store server 524 and an image reconstruction system 526. By way of example, the operator workstation 516, data store sever 524, and image reconstruction system 526 may be connected via a communication system 528, which may include any suitable network connection, whether wired, wireless, or a combination of both. As an example, the communication system 528 may include both proprietary or dedicated networks, as well as open networks, such as the internet.

The operator workstation 516 is also in communication with a control system 530 that controls operation of the CT system 500. The control system 530 generally includes an x-ray controller 532, a table controller 534, a gantry controller 536, and a data acquisition system 538. The x-ray controller 532 provides power and timing signals to the x-ray source 504 and the gantry controller 536 controls the rotational speed and position of the gantry 502. The table controller 534 controls a table 540 to position the subject 512 in the gantry 502 of the CT system 500.

The DAS 538 samples data from the detector elements 510 and converts the data to digital signals for subsequent processing. For instance, digitized x-ray data is communicated from the DAS 538 to the data store server 524. The image reconstruction system 526 then retrieves the x-ray data from the data store server 524 and reconstructs an image therefrom. The image reconstruction system 526 may include a commercially available computer processor, or may be a highly parallel computer architecture, such as a system that includes multiple-core processors and massively parallel, high-density computing devices. Optionally, image reconstruction can also be performed on the processor 522 in the operator workstation 516. Reconstructed images can then be communicated back to the data store server 524 for storage or to the operator workstation 516 to be displayed to the operator or clinician.

The CT system 500 may also include one or more networked workstations 542. By way of example, a networked workstation 542 may include a display 544; one or more input devices 546, such as a keyboard and mouse; and a processor 548. The networked workstation 542 may be located within the same facility as the operator workstation 516, or in a different facility, such as a different healthcare institution or clinic.

The networked workstation 542, whether within the same facility or in a different facility as the operator workstation 516, may gain remote access to the data store server 524 and/or the image reconstruction system 526 via the communication system 528. Accordingly, multiple networked workstations 542 may have access to the data store server 524 and/or image reconstruction system 526. In this manner, x-ray data, reconstructed images, or other data may be exchanged between the data store server 524, the image reconstruction system 526, and the networked workstations 542, such that the data or images may be remotely processed by a networked workstation 542. This data may be exchanged in any suitable format, such as in accordance with the transmission control protocol (“TCP”), the internet protocol (“IP”), or other known or suitable protocols.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for reconstructing an image of a subject using a computed tomography (CT) system, the steps of the method comprising:
(a) providing to a computer system, data acquired from a subject using a CT system;
(b) reconstructing a first image from the provided data using the computer system;
(c) generating a metal component mask from the first image using the computer system, wherein the metal component mask depicts regions in the subject containing metal;
(d) generating masked data with the computer system by using the metal component mask to remove ray sums in the provided data that pass through the regions in the subject containing metal;
(e) reconstructing a second image from the masked data using the computer system;
(f) generating a difference image with the computer system by computing a difference between the first image and the second image;
(g) generating corrected data with the computer system by forward projecting the difference image to generate segmented artifact data and by computing a difference between the provided data and the segmented artifact data; and
(h) reconstructing a third image from the corrected data using the computer system.

2. The method of claim 1, wherein the second image is reconstructed using a sinogram-sparsified iterative reconstruction (SSIR) that accounts for sparsity in the masked data.

3. The method as recited in claim 2, wherein the SSIR implements an iterative shrinking algorithm.

4. The method as recited in claim 1, wherein the first image is reconstructed using an analytical reconstruction.

5. The method as recited in claim 4, wherein the analytical reconstruction comprises a filtered backprojection.

6. The method as recited in claim 1, wherein the third image is reconstructed using an analytical reconstruction.

7. The method as recited in claim 6, wherein the analytical reconstruction comprises a filtered backprojection.

8. The method as recited in claim 1, wherein the metal component mask is generated by thresholding the first image using a threshold value that is associated with signal intensities corresponding to metal.

9. The method as recited in claim 1, wherein generating the masked data includes projecting the metal component mask into a sinogram domain.

10. The method as recited in claim 1, wherein generating the difference image includes computing a difference between the first image and the second image and then multiplying the difference by the metal component mask.

11. A computer system for reconstructing an image from data acquired with a computed tomography (CT) system, comprising:
one or more processors;
a memory having stored thereon instructions that when executed by the one or more processors cause the one or more processors to perform the steps comprising:
(a) accessing data acquired from a subject using a CT system;
(b) reconstructing a first image from the provided data;
(c) generating a metal component mask from the first image, wherein the metal component mask depicts regions in the subject containing metal;
(d) generating masked data by using the metal component mask to remove ray sums in the accessed data that pass through the regions in the subject containing metal;
(e) reconstructing a second image from the masked data;
(f) generating a difference image by computing a difference between the first image and the second image;
(g) generating segmented artifact data by forward projecting the difference image;
(h) generating corrected data by computing a difference between the accessed data and the segmented artifact data; and
(i) reconstructing a third image from the corrected data.

12. The computer system as recited in claim 11, wherein the one or more processors reconstruct the first image using an analytical reconstruction.

13. The computer system as recited in claim 12, wherein the one or more processors reconstruct the first image using a filtered backprojection.

14. The computer system as recited in claim 11, wherein the one or more processors reconstruct the second image using an iterative reconstruction that accounts for sparsity in the masked data.

15. The computer system as recited in claim 14, wherein the one or more processors reconstruct the second image using an iterative reconstruction that implements an iterative shrinking algorithm.

16. The computer system as recited in claim 11, wherein the one or more processors reconstruct the third image using an analytical reconstruction.

17. The computer system as recited in claim 16, wherein the one or more processors reconstruct the third image using a filtered backprojection.

18. The computer system as recited in claim 11, wherein the one or more processors generate the metal component mask by thresholding the first image using a threshold value that is associated with signal intensities corresponding to metal.

19. The computer system as recited in claim 11, wherein the one or more processors generate the masked data by projecting the metal component mask into a sinogram domain.

20. The computer system as recited in claim 11, wherein the one or more processors generate the difference image by computing a difference between the first image and the second image and then multiplying the difference by the metal component mask.
